(19) United States
(12) Patent Application Publication
(10) Pub. No.: US 2010/0055214 A1
(43) Pub. Date: Mar. 4, 2010

(54) METHOD FOR INCREASING BLOOD FLOW TO MAMMALIAN SKELETAL MUSCLE BY ADMINISTERING L-ARGNINE, CRATAEGUS EXTRACT, ARTICHOKE FLAVONOIDS AND GYMNOSTEMMA PENTAPHYLLUM EXTRACT

(75) Inventors: Marvin A. Heuer, Mississauga (CA); Shan Chaudhuri, Mississauga (CA); Ken Clement, Mississauga (CA); Michele Molino, Ontario (CA)

Correspondence Address: FITZPATRICK, CELLA, HARPER & SCINTO, 30 Rockefeller Plaza, New York, NY, 10112-3801, US

(73) Assignee: H3 FORMULATIONS LTD., Missisauga (CA)

(21) Appl. No.: 12/616,292

(22) Filed: Nov. 11, 2009

Related U.S. Application Data
(62) Division of application No. 11/945,588, filed on Nov. 27, 2007.
(60) Provisional application No. 60/868,855, filed on Dec. 6, 2006.

(51) Int. Cl. A61K 36/254; A61K 36/73; A61P 21/00

(52) U.S. Cl. 424/728; 424/765

(57) ABSTRACT

A nutritional composition and method is provided for increasing blood flow to skeletal muscle in an individual by supporting the biological activity of nitric oxide comprising therapeutically effective amounts of L-arginine, Crataegus extract and Artichoke flavonoids.

### CROSS REFERENCE TO RELATED APPLICATION

This application claims the benefit of U.S. Provisional Patent Application Ser. No. 60/868,855, filed Dec. 6, 2006, the content of which is incorporated by reference.

### FIELD OF THE INVENTION

The present invention relates to a nutritional composition and method for increasing blood flow in working skeletal muscle by supporting endogenous biological mechanisms.

### BACKGROUND OF THE INVENTION

The cardiovascular system provides blood flow to diverse tissues in a way analogous to the way a city water supply distributes water flow to diverse settings (Swain DP. The water-tower analogy of the cardiovascular system. Adv Physiol Educ. 2000 December; 24(1):43-50). The flow of blood within the cardiovascular system, just as the flow of water within a city water supply, must respond to a number of factors in order to meet the demands of the various tissues which may sometimes be competing for blood.

One such important factor is exercise, which induces signals for the increased blood flow requirement in order to meet the demands of working muscle tissue. The cardiac output to resting skeletal muscle in humans has been estimated at approximately 20% total cardiac output capacity which may increase up to 90% total cardiac output capacity during strenuous exercise (Delp MD, O'Leary DS. Integrative control of the skeletal muscle microcirculation in the maintenance of arterial pressure during exercise. J Appl Physiol. 2004 September; 97(3):1112-8). Increased blood flow to active skeletal muscle is important for increased nutrient import and waste export resulting from increased metabolism (Clark M G, Wallis M G, Barrett E J, Vincent M A, Richards S M, Clerk L H, Rattigan S. Blood flow and muscle metabolism: a focus on insulin action. Am J Physiol Endocrinol Metab. 2003 February; 284(2):E241-58). This adaptation and redistribution of blood flow relative to resting conditions is accomplished through a number of mechanisms,

In skeletal muscle, one such mechanism is mediated by endothelial cells (Griendling K K, Alexander R W. Endothelial control of the cardiovascular system: recent advances. FASEB J. 1996 February; 10(2):283-92). Endothelial cells form the endothelium which is a layer of cells lining the inner side of blood vessels. Comprising the outer lining of blood vessels is a layer of vascular smooth muscle. Endothelial cells synthesize and release nitric oxide (NO). NO then provides signals which result in a widening of blood vessels, also known as vasodilation, by signaling vascular smooth muscle to relax.

The oxidation of L-arginine by the enzyme NO synthase (NOS) results in the production of NO. All major nitric oxide synthase (NOS) isoforms and splice variants, including a muscle-specific splice variant, are expressed in the skeletal muscles of all mammals (Stamler J S, Meissner G. Physiology of nitric oxide in skeletal muscle. Physiol Rev. 2001 January; 81(1):209-237).

Thus, in muscles, NO is a signaling molecule which increases blood flow, thereby increasing nutrient uptake and waste excretion by metabolically active muscles. As such in terms of athletic performance with respect to skeletal muscle it would be advantageous to increase and sustain levels of NO. Furthermore, it would be advantageous to expedite the increase of NO levels and activity through a rapid delivery of NO-modulating compositions.

### SUMMARY OF THE INVENTION

The foregoing needs and other needs and objectives that will become apparent for the following description are achieved in the present invention, which comprises a nutritional supplement and method for facilitating the vasodilation-action of NO in skeletal muscle. Said composition provides a source of an effective amount of L-arginine or derivatives thereof, an effective amount of Crataegus extract, an effective amount of Artichoke flavonoids. The composition acts to jointly and simultaneously support, facilitate or otherwise enhance the activity of endogenous nitric oxide leading to increased vasodilation in skeletal muscle.

### DETAILED DESCRIPTION OF THE INVENTION

In the following description, for the purposes of explanations, numerous specific details are set forth in order to provide a thorough understanding of the present invention. It will be apparent, however, to one of ordinary skill in the art that the present invention may be practiced without these specific details.

The present invention is directed towards a nutritional supplement and method to facilitate and encourage the vasodilation of blood vessels in skeletal muscle through NO-dependent mechanisms.

Endogenous NO functions include the signaling of vasodilation of blood vessels. Vasodilation, thus in turn leads to increased blood flow, particularly in working skeletal muscle, wherein an increase in the transport of nutrients and waste products is achieved. Both nutrient requirements and waste products increase with increasing muscle metabolism. Therefore, increased vasodilation can advantageously assist in the transport of skeletal muscle requirements and metabolic products during periods of exercise.

The endogenous activity of NO function can be supported, facilitated, or otherwise enhanced by a number of mechanisms including but not limited to: increased synthesis of NO by increased precursor availability, increased NO synthesis due to enhanced NOS activity or increased NO synthesis due to increased NOS gene transcription. Various amalgamations of the aforementioned mechanisms will ensure a constant supply of NO during periods of skeletal muscle exercise.

In a preferred embodiment of the present invention, a composition comprising L-arginine or derivatives thereof, Crataegus extract and Artichoke flavonoids is provided to support, facilitated, or otherwise enhance a number of the above mechanisms involving the endogenous activity of NO functionality.

L-arginine

L-arginine (CAS No. 74-79-3) is considered a semi-essential amino acid. Normally L-arginine is synthesized in sufficient amounts by the body. However, conditions and circumstances are known wherein additional L-arginine supplementation is required.

As a precursor to NO, L-arginine plays an important role in regulating cardiovascular endothelium-dependent processes. Many of the therapeutic effects of L-arginine are likely due to its role as a NO precursor (Appleton J. Arginine: Clinical potential of a semi-essential amino. Altern Med Rev. 2002 December; 7(6):512-22). Additionally, L-arginine has been shown to increase aerobic exercise capacity and NO production (Maxwell A J, Ho H V, Le C Q, Lin P S, Bernstein D, Cooke J P. L-arginine enhances aerobic exercise capacity in association with augmented nitric oxide production. J Appl Physiol. 2001 March; 90(3):933-8).

Nitric oxide is a free radical which generated in biological systems. Nitric oxide synthase enzymes produce NO through the catalysis of a five-electron oxidation of the guanidine nitrogen of L-Arginine. In this process, L-Arginine is oxidized to L-Citrulline via two successive monoxygenation reactions. In the first reaction, NOS acts with NADPH and O₂ to produce N^{ω}hydroxy L-Arginine as an intermediate in the overall reaction. N^{ω}hydroxy L-Arginine is then further oxidized to form L-Citrulline and NO. The reaction takes places in the endothelial cells where Ca++-calmodulin, NADPH, tetrahydrobiopterin, FAD and FMN are required as co-factors.

U.S. Pat. No. 5,945,452 discloses the use of orally administered L-arginine or its physiologically acceptable salts, in an amount sufficient to enhance the level of endothelial nitric oxide to inhibit the development of atherosclerosis, restenosis or thrombosis in the vascular system.

U.S. Pat. No. 6,340,480 discloses a composition comprising an effective amount of L-arginine, ginseng and Ziyphi fructus being administered to stimulate release of NO for the promotion of circulation.

In an embodiment of the present invention, which is set forth in greater detail in the examples below, the nutritional supplement includes L-arginine or derivatives thereof. A serving of the nutritional supplement may include from about 1.0 g to about 3.0 g of L-arginine or derivatives thereof. The preferred dosage of a serving of the nutritional supplement comprises about 2.0 g of L-arginine or derivatives thereof.

Crataegus Extract

Crataegus, also called hawthorn, is an herb in Traditional Chinese Medicine used in formulations for strengthening heart function, lowering blood lipids, and dilating blood vessels to promote blood circulation. Extracts of Crataegus have demonstrated efficacy at treating cardiovascular-related conditions such as congestive heart failure (Degenring F H, Suter A, Weber M, Saller R. A randomized double blind placebo controlled clinical trial of a standardized extract of fresh Crataegus berries (Crataegisan) in the treatment of patients with congestive heart failure NYHA II. Phytomedicine. 2003; 10(5):363-9 Abstract only) and hypertension (Asgary S, Naderi G H, Sadeghi M, Kelishadi R, Amiri M. Antihypertensive effect of Iranian Crataegus curvisepala Lind.: a randomized, double-blind study. Drugs Exp Clin Res. 2004; 30(5-6):221-5 Abstract only). Furthermore, Crataegus extract has been shown to induce endothelial-dependant vasodilation (Kim S H, Kang K W, Kim K W, Kim N D. Procyanidins in crataegus extract evoke endothelium-dependent vasorelaxation in rat aorta. Life Sci. 2000; 67(2):121-31 Abstract only) via the phosphorylation of endothelial NO synthase (Brixius K, Willms S, Napp A, Tossios P, Ladage D, Bloch W, Mehlhorn U, Schwinger R H. Crataegus special extract WS 1442 induces an endothelium-dependent, NO-mediated vasorelaxation via eNOS-phosphorylation at serine 1177. Cardiovasc Drugs Ther. 2006 June; 20(3):177-84 Abstract only).

In an embodiment of the present invention, which is set forth in greater detail in the examples below, the nutritional supplement includes Crataegus extract. A serving of the nutritional supplement may include from about 0.0001 g to about 0.0050 g of Crataegus extract. The preferred dosage of a serving of the nutritional supplement comprises about 0.0010 g of Crataegus extract.

Artichoke Flavonoids

Artichoke (Cynara scolymus L.) is an ancient medicinal plant traditionally used primarily as a digestive aid. Artichoke flavonoids have been shown to induce an increase in endothelial NOS gene transcription and NO production in human endothelial cells (Li H, Xia N, Brausch I, Yao Y, Forstermann U. Flavonoids from artichoke (Cynara scolymus L.) up-regulate endothelial-type nitric-oxide synthase gene expression in human endothelial cells. J Pharmacol Exp Ther. 2004 September; 310(3):926-32).

In an embodiment of the present invention, which is set forth in greater detail in the examples below, the nutritional supplement includes Artichoke flavonoids. A serving of the nutritional supplement may include from about 0.0001 g to about 0.0050 g of Artichoke flavonoids. The preferred dosage of a serving of the nutritional supplement comprises about 0.0010 g of Artichoke flavonoids.

In another embodiment of the present invention, in addition to L-arginine or derivatives thereof, Crataegus extract and Artichoke flavonoids, the composition also may include one or more of: Xanthinol Nicotinate, L-norvaline or derivatives thereof, Asian Ginseng Powder root extract, French Maritime Pine Bark Extract, Citrulline or derivatives thereof, Gymnostemma Pentaphyllum, and Salvia miltiorrhiza (Cryptotanshinone). Xanthinol Nicotinate, L-norvaline or derivatives thereof, Asian Ginseng Powder root extract, French Maritime Pine Bark Extract, Citrulline or derivatives thereof, Gymnostemma Pentaphyllum, and Salvia miltiorrhiza (Cryptotanshinone) are additionally provided to support, facilitated, or otherwise enhance a number of mechanisms involving the endogenous activity of NO functionality.

The composition of the present invention described herein supports and facilitates the production of NO as well as its endogenous biological activity. L-arginine serves as a precursor of NO synthesis; flavonoids from Artichoke increase the transcription of the gene for endothelial NOS, accompanied by increased NO production; and Crataegus extract induces increased activity of endothelial NOS.

According to various embodiments of the present invention, the nutritional supplement may be consumed in any form. For instance, the dosage form of the nutritional supplement may be provided as, e.g., a powder beverage mix, a liquid beverage, a ready-to-eat bar or drink product, a capsule, a liquid capsule, a tablet, a caplet, or as a dietary gel. The preferred dosage form of the present invention is as a powder.

Furthermore, the dosage form of the nutritional supplement may be provided in accordance with customary processing techniques for herbal and nutritional supplements in any of the forms mentioned above. Additionally, the nutritional supplement set forth in the example embodiment herein may contain any appropriate number and type of excipients, as is well known in the art.

The present nutritional composition or those similarly envisioned by one of skill in the art, may be utilized in methods to support, facilitate or otherwise enhance the activity of endogenous NO leading to increased vasodilation in skeletal muscle. In particular, the present nutritional composition may be utilized to increase vasodilation of skeletal muscle during times of strenuous physical exercise leading to increased blood flow, whereby enhanced nutrient and waste transport is achieved.

In various embodiments of the present invention, the composition or portion thereof is provided in the form of fine-milled particles. As used herein, the terms “fine-milled” and/or “fine-milling” refer the process of micronization. Micronization is a mechanical process which involves the application of force to a particle, thereby resulting in a reduction in the size of said particle. U.S. Provisional Application No. 60/776,325 entitled “Compositions and Method for Increasing Bioavailability of Compositions for Performance Improvement”, which is herein fully incorporated by reference discloses a method of improving the absorption, palatability, taste, texture and bioavailability of compounds by increasing the solubility. The increased bioavailability of a compound or ingredients is achieved via a reduction in particle size using a “fine-milling” technique. Any acceptable fine-milling technique wherein the result is the fine-milled particles having an average particle size of between about 50 microns to about 2 microns. The reduction in size of the particles increases the surface area-to-volume ratio of each particle, thus increasing the rate of dissolution, thereby improving the rate of absorption.

Although the following example illustrates the practice of the present invention in one of its embodiments, the example should not be construed as limiting the scope of the invention. Other embodiments will be apparent to one of skill in the art from consideration of the specifications and example.

### EXAMPLE

A nutritional supplement is provided in one serving per day in powder form. A single serving of the nutritional supplement comprises from about 1.0 g to about 3.0 g of L-arginine or derivatives thereof, from about from about 0.0001 g to about 0.0050 g of Cartages extract and from about 0.0001 g to about 0.0050 g of Artichoke flavonoids.

Directions: As a nutritional supplement, at least one serving of the powder is provided daily, up to three servings per day. Said servings are mixed with 8 oz. of water and consumed at least once daily. Each serving may be consumed immediately before exercise.

1. (canceled)

2. A method for increasing blood flow to skeletal muscle in a mammal, comprising the step of:
administering to said mammal an effective amount of a composition comprising L-arginine or derivatives thereof, Crataegus extract, Artichoke flavonoids and Gymnostemma pentaphyllum extract,
whereby said composition acts to enhance the activity of endogenous nitric oxide leading to increased vasodilation in skeletal muscle.

3. (canceled)

4. The method of claim 2, wherein said composition further comprises one or more of the group consisting of:
Xanthinol Nicotinate, L-norvaline or derivatives thereof, Asian Ginseng Powder root extract, French Maritime Pine Bark Extract, Citrulline or derivatives thereof, and Salvia miltiorrhiza.

5. A method for increasing blood flow to skeletal muscle in a mammal, comprising the step of administering to said mammal an effective amount of a composition consisting essentially of L-arginine or derivatives thereof, Crataegus extract, Artichoke flavonoids and Gymnostemma pentaphyllum extract,
whereby said composition acts to enhance the activity of endogenous nitric oxide leading to increased vasodilation in skeletal muscle.

6. A method for increasing blood flow to skeletal muscle in a mammal, comprising the step of administering to said mammal an effective amount of a composition consisting essentially of L-arginine or derivatives thereof, Crataegus extract, Artichoke flavonoids Gymnostemma pentaphyllum extract, and
one or more of the group consisting of Xanthinol Nicotinate, L-norvaline or derivatives thereof, Asian Ginseng Powder root extract, French Maritime Pine Bark Extract, Citrulline or derivatives thereof, and Salvia miltiorrhiza,
whereby said composition acts to enhance the activity of endogenous nitric oxide leading to increased vasodilation in skeletal muscle.
